(19) United States
(12) Patent Application Publication
(10) Pub. No.: US 2011/0262562 A1
(43) Pub. Date: Oct. 27, 2011

(54) AQUEOUS SALINE SOLUTIONS FOR THE DESTRUCTION OF FATTY TISSUE

(75) Inventors: Yves Crassas, Lyon (FR); Denis Blache, Messigny et Vantoux (FR)

(73) Assignee: LIPOLYANE, Tassin La Demi Lune (FR)

(21) Appl. No.: 13/126,318

(22) PCT Filed: Oct. 27, 2009
(86) PCT No.: PCT/FR2009/052063

§ 371 Date: Jul. 6, 2011

(30) Foreign Application Priority Data
Oct. 27, 2008 (FR) 08/05960

(51) Int. Cl. A61K 33/14; A61P 17/00; A61Q 19/06; A61Q 19/00; A61K 33/00; A61K 8/19

(52) U.S. Cl. 424/680; 424/722; 424/717; 424/681

(57) ABSTRACT

The invention relates to aqueous saline solutions with an osmolarity of 50 mOsm/L to 170 mOsm/L, which further include at least one membrane-weakening agent. The invention also relates to a cosmetic composition which includes one of said solutions, as well as to the use of one of said solutions to obtain a drug for treating cellulite, steatomery, and lipomas.

### TECHNICAL FIELD

The present invention relates to aqueous saline solutions which may be used for destroying fatty tissues, in particular hypotonic solutions.

### BACKGROUND

In spite of particular dietetic care and/or regular sporting activity, an unsightly accumulation of fat (currently designated as cellulitis) may be seen in certain persons. This trend is the consequence of changes in lifestyle, also leading to a general increase in overweight or even obesity by localized accumulation of stored fats.

In this respect, within the scope of the present invention, the term of <<cellulitis>> designates, according to currents parlance, an aesthetical cellulitis, i.e. hypertrophy of adipous tissue generally highly localized forming nodes called <<orange peel skin>>.

Cellulitis is actually surface hyperlipodystrophy while steatomery corresponds to localized and deeper hyperlipodystrophy.

It should be noted that from an anatomic point of view, there exists under the skin (which consists of the epidermis and of the dermis) three layers of fat:
the most superficial layer, located in the hypodermis, is organized in fatty chambers separated from each other by elastic connective partitions. This layer of <<hypodermic fat>>, the metabolism of which is mainly under hormonal control, is responsible for the occurrence of cellulitis; and then
under the hypodermis, two layers of stored fat, the metabolism of which is essentially under control of genetics and lifestyle (diet, physical activity), i.e.:
   <<parallel fat>> which has variable thickness depending on the corpulence of the patient. This fat is particularly sensitive to diet,
   a still deeper fat, called <<steatomeric fat>> or <<structural fat>>. Its distribution on the body is irregular and varies according to the individuals. This fat is very resistant to diet and is responsible for irregularities of the figure, like saddlebags.

One of the present techniques for treating significant accumulation of adipous tissue is liposuction, a highly practiced surgical operation, but which is not without risks, in particular vital risks for obese patients. Liposuction actually requires long anaesthesia and leaves a large number of aesthetically damaging scars. It is therefore easy to understand the fad for non-surgical techniques, in particular for reducing the presence of localized excessive adipous tissues.

Adipocytolysis presently represents one of these non-surgical techniques. It is used as a definitive non-surgical treatment of steatomeries, but also of aesthetical cellulitis, acting directly on the adipous tissue by breaking the cell membrane of adipocytes.

This technique is based on the effect of osmotic pressure, the measurement physical unit of which is the osmole (Osm), expressed in moles per liter. Thus, when a mole of substance is dissolved in a liter of water, it exerts an osmotic pressure of one osmole. Osmolarity is the concentration of dissolved substance exerting osmotic power. For example, the osmolarity of plasma is considered as being comprised between 280 mOsm/L and 330 mOsm/L, that of the cell cytoplasm is 394 mOsm/L. Thus, the terms of “hypotonia”, “isotonia” and “hypertonia” are used herein for respectively designating a compartment, the osmolarity of which is less than, equal to and greater than the osmolarity of the plasma.

More specifically, the lytic effect of this adipocytolysis technique is obtained by the combination of physico-chemical and biophysical effects. First of all, it begins by the intra-fat injection of a strongly hypotonic solution (between 60 mOsm/L and 90 mOsm/L). This injection generates an osmotic pressure difference on either side of the (semi-permeable) membrane of the adipocytes, which causes their hyperhydration, because of a flow of liquid from the compartment with the lowest osmolarity (the interstitial liquid) towards the compartment of greater osmolarity (the cytoplasm of the adipocytes). This leads to an increase in the volume of the adipocytes, as well as to embrittlement of their membrane, or even to cell lesions. Destruction of the adipocytes is then completed by a light biophysical treatment, such as the application of a transcutaneous ultrasonic field. The lesion and then the destruction of the adipocytes cause salting out of the fats, as well as of triglycerides, free fatty acids and other cell contents into the plasma, which will be degraded by the liver in order to be eliminated. The cell debris are managed by the monocytes and macrophages.

Patent FR 2 876 907 A1 attempts to find a remedy to the drawbacks which are in particular those of the repetition of injections of highly hypotonic solutions, those of recourse to complementary liposuction, and this by administration of an iso- or hypo-tonic and hyperkaliemic solution, prior to the aforementioned aesthetical treatment by adipocytolysis. This has the advantage of reducing the volume of the adipocytes, and thus of targeting a maximum number of cells at a time during the step for intra-fat injection of the highly hypertonic solution. This patent further discloses synergy of action between a hyperkaliemic isotonic solution, or a hypotonic solution, possibly hyperkaliemic, with certain substances such as tiratricol (triiodothyroacetic acid) or an amphoteric compound such as phosphatidylcholine.

Nonetheless, one of the major problems of this adipocytolysis technique remains the optimization of the composition of the highly hypotonic solutions. Indeed, the injection of hypotonic solutions, or even highly hypotonic solutions, may increase the volume of the adipocytes but without necessarily leading to their bursting, thereby making the adipocytolysis treatment inefficient.

### BRIEF SUMMARY

This is why the present invention proposes to solve the problem of efficient lysis of the membranes of the adipocytes during treatment of steatomery, of cellulitis, as well of lipoma which consists in a treatment by adipocytolysis.

The inventors have developed aqueous saline solutions having osmolarity comprised between 50 mOsm/L and 170 mOsm/L, preferably between 50 mOsm/L and 100 mOsm/L, which further comprise at least one membrane-weakening agent.

Within the scope of the present invention, by membrane-weakening agent is meant a compound which is suitable for exerting a weakening action on the membrane of cells, in particular of adipocytes. Said compounds have to be powerful solubilizers of the bilayer of lipids of which the membranes of cells are made up. To do this, these compounds are inserted into the bilayer of lipids so as to destabilize it by generating pores and to disorganize it; which has the consequence of cell lysis. The following publications:
“Current techniques for single-cell lysis”, J.R. Soc. Interface (2008) 5, S131-S138, of BROWN et al.
“Mixed micelles and other structures in the solubilization of bilayer lipid membranes by surfactants” Biochem Biophys Acta (2000); 146-63, of ALMGREN,
   more fully detail the mode of action of these membrane-weakening agents described above.

The membrane-weakening agent may be selected from ionic detergents, non-ionic detergents, zwitterionic detergents and derivatives of glycerol.

As examples, among non-ionic detergents, mention may notably be made of:
alkylglycosides such as n-octyl beta D-glucopyranoside,
glucamides, such as N,N′-bis(3-D-gluconamido-propyl)cholamide, known under the trade name of Big CHAP,
polyoxyethylenes such as poloxamers, 2,3-dihydroxypropyloctanoate (TWEEN® 20), 2-[4-(2,2,4-trimethylpentan-2-yl)phenoxy]ethanol (TRITON® X 100), 2-dodecoxyethanol (Brij® 35).

Among ionic detergents, mention may notably be made of salts of biliary acids and their derivatives, as well as salts of alkylammonium, fatty acid derivatives of amino acids, carnitines, glyceride derivatives of amino acids, acyl lactylates, mono-, di-acetylated tartaric acid esters of mono- and di-glycerides, succinoyl monoglycerides, citric acid esters of mono- and of di-glycerides, alginate salts, propylene glycol alginate, lecithins and hydrogenated lecithins, lysolecithins, and hydrogenated lysolecithins, lysophospholipids and their derivatives, phospholipids and their derivatives, alkylsulfate salts, fatty acid salts, docusate sodium.

Among the zwitterionic detergents, mention may notably be made of 3-[(3-cholamidopropyl)-dimethylammonio]-1-propane-sulfonate, N-tetradecyl-N,N-dimethyl-3-ammonium-1-propane-sulfonate, lauryl dimethyl-betaine.

The inventors have found that the adjunction to aqueous saline solutions with osmolarity comprised between 50 mOsm/L and 170 mOsm/L of these different membrane-weakening agents has a weakening action on the membrane of adipocytes, and more particularly when the osmolarity is 60 mOsm/L. Thus, these aqueous saline solutions give the possibility of promoting lysis of adipocytes upon their intra-fat injection during treatment of cellulitis, steatomery, as well as of lipoma which may consist in a treatment by adipocytolysis.

Preferably, salts of biliary acids and poloxamers are used as membrane-weakening agents; poloxamers being most preferred.

The inventors have discovered the synergistic effect between biliary acid salts and/or poloxamers in solutions with osmolarity comprised between 50 and 170 mOsm/L in order to promote lysis of adipocytes, and more particularly when the osmolarity is 60 mOsm/L.

The biliary acid salts form a large family of molecules, having the structure of a steroid (i.e. a tetra-cyclic hydrocarbon) including a side chain comprising five to eight carbon atoms and ending with a carboxylate function.

Within the scope of the present invention, the biliary acid salts may be selected from the following salts: sodium deoxycholate (DC), sodium tauro-deoxycholate (TDC), sodium lithocholate (LC) and sodium tauro-lithocholate (TLC), or further the salt of cholic acid, the salt of chenodeoxycholic acid, the salt of 7-alpha-dehydroxylate chenodeoxycholic acid, the salt of ursodeoxycholic acid, the salt of dihydroxytauric acid, the salt of trihydroxytauric acid, and the salts of their glycine conjugates.

Preferably, the TDC or TLC is selected as a biliary acid salt.

The biliary acid salts are present in the aqueous saline solutions according to the invention at a concentration comprised between 50 μM and 3,000 μM, preferably between 50 μM and 1,000 μM, even more preferentially between 150 μM and 750 μM.

The structure of poloxamers (also known under the trade name of Pluronic® copolymer blocks, marketed by BASF) added to the aqueous saline solutions according to the invention is described in the publication of A. KABANOV, entitled “Pluronic® block copolymers: novel functional molecules for gene therapy”, published in Advanced Drug Delivery Reviews 54 (2002) 223-233. These are ethylene oxide (EO) and propylene oxide (PO) blocks arranged according to the following tri-block structure: EOₓ—PO_{y}-EOₓ.

This arrangement leads to an amphiphilic copolymer, wherein the number of hydrophilic units EO(x) and the number of hydrophobic units PO(y) may be modified in order to vary in size, in hydrophilicity and lipophilicity. These poloxamers known for their absence of toxicity, are customarily used for their surfactant properties.

Subsequently, will be meant by:
<<hydrophilic portion of the poloxamer>>: the whole of the EO blocks of said poloxamer,
<<hydrophobic portion of the poloxamer>>: the whole of the PO blocks of said poloxamer.

The structural formula of the poloxamers is the following (I):

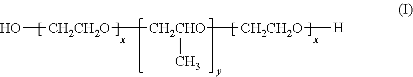

A formula wherein x and y represent integers.

In the formula (I) above:
the block CH₂CH₂O is therefore the block designated as EO hereinbefore,
the group CH₂CHO is therefore the block designated

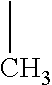

as PO hereinbefore,

Examples of poloxamers may be the following compounds, available at BASF:
Pluronic® L61 EO₂—PO₃₀-EO₂MW=1,950,
Pluronic® P85 EO₂₆—PO₄₀-EO₂₆ MW=4,600.

The nomenclature of these Pluronic® poloxamers is detailed in appendix A of the aforementioned KABANOV publication. The letters <<F>>, <<L>>, <<P>>, respectively indicate a solid, liquid or pasty state of the poloxamer. The indicated numerical figures define the structural parameters of these polymers. Thus, the last figure approximately indicates the weight content of the hydrophilic portion of the poloxamer, expressed in <<tens>>. For example, if the last figure is 8, this means that the hydrophilic portion represents 80% by weight of the poloxamer. The other remaining figures (one or two) are indicative of the molar mass of the hydrophobic portion of the poloxamer. For this, the idea is to multiply the corresponding number by 300 in order to obtain the approximate molecular mass expressed in Da.

As a nomenclature example, the poloxamer F 127 is in the solid state, the mass of the hydrophobic portion of this poloxamer is 3,600 Da (12×300) and the hydrophilic portion represents 70% by weight of the poloxamer.

According to the publication of I. R. SCHMOLKA, entitled <<A review of block polymer surfactants>>, and published in J. Am. Chem. Oil Chem. Soc. 54 (1977) 110-116, the poloxamers may be synthesized by sequential polymerization of PO and EO monomers, in the presence of an alkaline catalyst, such as sodium or potassium hydroxide.

Within the scope of the present invention, at least one poloxamer of structural formula (I) as defined above may be used, comprising a hydrophilic portion and a hydrophobic portion, x and y being selected so that the hydrophilic portion represents 10 to 80% by weight of the poloxamer, preferably 30 to 50%, and so that the HLB (“hydrophilic/lipophilic balance”) is less than 35, preferably less than 20. The HLB values are determined by the procedure described in the publication J. Am. Oil Chem. Soc. 41(1964): 169, the authors of which are BECHER, P. and BIRKMEIER R. L., which applies column chromatography, the solvent of which is a mixture of n-hexane and ethanol.

Thus, within the scope of the present invention, the poloxamers marketed by BASF which are Pluronic®: L31, L35, F38, L42, L43, L44, L61 to L64, L72, P75, F77, L81, P75, P84, P85, F87, F88, L92, F98, L101, P103 to P105, F108, L121, L122, P123 may be present in the aqueous saline solutions according to the invention, and the characteristics of which in % by weight of the hydrophilic portion and the HLB are shown in the Table hereafter:

TABLE 1
characteristics of the hydrophilic portion % and
of the HLB of poloxamers.

| Pluronic ® | HLB | % of hydrophilic portion |
|---|---|---|
| L31 | 5 | 10 |
| L35 | 19 | 50 |
| F38 | 31 | 80 |
| F68 | 29 | 80 |
| L42 | 8 | 20 |
| L43 | 12 | 30 |
| L44 | 16 | 40 |
| L61 | 3 | 10 |
| L62 | 7 | 20 |
| L63 | 11 | 30 |
| L64 | 15 | 40 |
| L72 | 17 | 20 |
| P75 | 14 | 50 |
| P84 | 14 | 40 |
| P85 | 16 | 50 |
| F87 | 24 | 70 |
| F88 | 28 | 80 |
| L92 | 6 | 20 |
| F98 | 28 | 80 |
| L101 | 1 | 10 |
| P103 | 9 | 30 |
| P104 | 13 | 40 |
| P105 | 15 | 50 |
| F108 | 27 | 80 |
| L121 | 1 | 10 |
| L122 | 4 | 20 |
| P123 | 8 | 30 |

The invention relates to an aqueous saline solution as described above, wherein the poloxamer is selected from poloxamers of formula (I) which have:
a hydrophilic portion representing 40% of poloxamer and the HLB is 16,
a hydrophilic portion representing 10% of poloxamer and the HLB is 3,
a hydrophilic portion representing 20% of the poloxamer, and the HLB is 7,
a hydrophilic portion representing 30% of the poloxamer, and HLB 11 ,
a hydrophilic portion representing 40% of the poloxamer and the HLB is 15,
a hydrophilic portion representing 50% of the poloxamer and the HLB is 14,
a hydrophilic portion representing 40% of the poloxamer and the HLB is 14, and
a hydrophilic portion representing 50% of the poloxamer and the HLB is 16,
a hydrophilic portion representing 30% by weight of the poloxamer and the HLB is 8.

Suitably, Pluronic® poloxamers are preferably used:
L44, the hydrophilic portion of which represents 40% by weight of the poloxamer and the HLB is 16,
L61, the hydrophilic portion of which represents 10% by weight of the poloxamer and the HLB is 3,
L62, the hydrophilic portion of which represents 20% by weight of the poloxamer and the HLB is 7,
L63, the hydrophilic portion of which represents 30% by weight of the poloxamer and the HLB is 11,
L64, the hydrophilic portion of which represents 40% by weight of the poloxamer and the HLB is 15,
P75, the hydrophilic portion of which represents 50% by weight of the poloxamer and the HLB is 14,
P84, the hydrophilic portion of which represents 40% by weight of the poloxamer and the HLB is 14,
P85, the hydrophilic portion of which represents 50% by weight of the poloxamer and the HLB is 16
P123, the hydrophilic portion of which represents 30% by weight of the poloxamer and the HLB is 8.

According to the present invention, the Pluronic® P85, L61 and 64 poloxamers are preferred poloxamers. Advantageously, a poloxamer of formula (I) is used which has a hydrophilic portion representing 30% by weight of the poloxamer and the HLB is 8, and preferably Pluronic® P123 is used.

The poloxamers may be present in the aqueous saline solutions according to the invention at a concentration comprised between 0.001% and 5% by weight/volume, preferably between 0.1% and 1%.

The invention is directed toward an aqueous saline solution having osmolarity comprised between 50 mOsm/L and 170 mOsm/L, preferably between 50 mOsm/L and 100 mOsm/L, which comprises at least one biliary acid salt.

The invention further provides an aqueous saline solution having osmolarity comprised between 50 mOsm/L and 170 mOsm/L, preferably between 50 mOsm/L and 100 mOsm/L, which comprises at least one poloxamer.

Additionally, the invention provides an aqueous saline solution having osmolarity comprised between 50 mOsm/L and 170 mOsm/L, preferably between 50 mOsm/L and 100 mOsm/L, which comprises at least one poloxamer and at least one biliary acid salt.

From WO 2005/063169 A2 and from WO 2005/041919 A2, aqueous solutions are known, containing at least one phospholipid and/or at least biliary acid which may be used in the preparation of drugs intended for treating accumulation of adipous tissues. It is further specified that these solutions may be diluted in a saline solution. The osmolarity of the aqueous solutions is by no means specified in these documents, in particular the requirement that the solution be hypotonic is by no means mentioned. However, it is known in the medical field that a saline solution (for example an NaCl solution at a concentration of 9 g/L) may consist in a physiological liquid, i.e. a liquid having osmolarity similar to the main body fluids, such as blood, therefore of the order of 300 mOsm/L.

Within the scope of the present invention, the aforementioned aqueous saline solutions may be solutions essentially containing NaCl, NaHCO₃, MgCl₂ salts, having concentrations which may be respectively comprised between 10 and 25 mmol/L, 5 and 15 mml/L and 0.5 and 5 mmol/L.

Further, the aqueous saline solutions according to the invention are without any potassium salts. Indeed, the presence of potassium may prove to be painful and dangerous, in particular in patients having cardiopathy.

Further, preferably, the aqueous saline solution according to the invention contains magnesium; this element giving the possibility of combating an oxidizing and inflammatory stress which may occur during treatment by adipocytolysis.

Within the scope of the present invention, other substances may be present in the aqueous saline solutions according to the invention or added extemporaneously. These may be adrenalin, and an anaesthetic such as lidocaine.

Adrenalin may have a concentration comprised between 0.5 and 2 mg/L. The anaesthetic such as lidocaine may have a concentration comprised between 1 and 5% by weight/volume.

Preferably, the aqueous saline solutions according to the invention comprise adrenalin at a concentration of 1 mg/L and lidocaine in an amount of 2% by weight/volume.

The mixture of these substances with biliary acids and poloxamers is quite appreciable. Indeed, synergy is observed between these compounds and the aforementioned substances, by which it is possible to reduce the concentrations of the compounds which are the biliary acid salts and the poloxamer. The biliary acid salt concentration was thereby able to be reduced by about 50% in order to obtain the same lysis effect on adipocytes.

The invention is also directed toward the use of an aqueous saline solution according to the invention as a drug.

The present invention also relates to the non-therapeutic use of an aqueous saline solution according to the invention for producing lysis of adipocytes.

The present invention also relates to a cosmetic composition characterized in that it comprises an aqueous saline solution according to the invention.

The invention also relates to the use of a cosmetic composition according to the invention for non-therapeutic treatment of cellulitis, of steatomery as well as lipoma.

Moreover, the invention also relates to the use of an aqueous saline solution according to the invention for obtaining a drug intended for treating cellulitis, steatomery as well as lipoma.

The present invention also relates to a medical device for treating cellulitis, steatomery as well as lipoma.

The present invention also relates to a method for aesthetical treatment consisting in the administration of an aqueous saline solution according to the present invention, in order to be able to in particular act on cellulitis, steatomery, as well as on lipoma. Said administration may be performed by an intra-fat injection method. The administration of the aqueous saline solution according to the invention may be performed in all the fat layers described hereinbefore, i.e.:
hypodermal fat,
parallel fat, and
steatomeric fat.

Preferably, administration of the aqueous saline solution according to the invention is carried out in the steatomeric fat layer which is the deepest layer.

This is particularly advantageous, since this deep fat layer may be quite affected and therefore treated by administration of the aqueous saline solution according to the invention, while it is not very sensitive to dietetic treatments.

Thus, the aesthetical treatment method according to the present invention is particularly suitable for layers of fats, such as the steatomeric fat layer, for which customary diet methods remain inefficient, and therefore for which no valid method was presently available for destroying these deep adipous tissues.

The aesthetical treatment method according to the invention gives the possibility of providing efficient and relatively durable correction of the figure.

The present invention relates to an aesthetical treatment method consisting in the intra-fat injection of an aqueous saline solution according to the invention, i.e. an aqueous saline solution of osmolarity which may be comprised between 50 and 170 mOsm/L, preferably comprised between 50 and 100 mOsm/L, and more preferably 60 mOsm/L, and which may comprise in addition to at least one biliary acid salt and/or at least one poloxamer as described above, at least one of the substances such as adrenalin or an anaesthetic such as lidocaine.

In an embodiment of the invention, the administration may be performed in the following way:
after local anaesthesia by small epidermal injections of the solution according to the invention containing lidocaine (of the order of 100 μL),
the solution according to the invention is injected subcutaneously into the targeted adipous tissue, and this at different injection depths, but always through the same injection point.

Thus, up to about a final volume of one liter may be administered by injection, and distributed homogeneously in the areas to be treated which may be the thighs, the hips, the abdomen or further the arms.

More specifically, after prior location of the areas to be treated by concentric curves and injection points, said injections are carried out over a period which may spread from 20 to 30 minutes, by a manual or optionally automatic method.

The non-therapeutic aesthetical treatment method according to the invention, in order to be quite effective, may be completed by an ultrasonic treatment (unfocussed ultrasound at low frequencies, preferably at 0.4 MHz) for a duration of 15 to 20 minutes, by means of a specially adapted apparatus. Better results may thereby be obtained as regards the aesthetical treatment according to the invention.

In another embodiment of the invention, substances such as adrenalin or an anaesthetic such as lidocaine are not present in the aqueous saline solution according to the invention, but may be mixed extemporaneously with an aqueous saline solution according to the invention at the intra-fat injection.

The examples hereafter will allow illustration of the present invention without however limiting its scope.

### BRIEF DESCRIPTION OF THE DRAWINGS

FIG. 1 is a graph illustrating the diameter of incubated adipocytes in the presence of aqueous saline solutions with osmolarities: 300 mOsm/L, 150 mOsm/L, 90 mOsm/L versus time.

FIG. 2 is a graph illustrating the time-dependent change in the number of adipocytes versus time for incubated adipocytes in aqueous saline solutions with osmolarities of 90, 150 and 300 mOsm/L.

FIG. 3 is a graph illustrating the diameter of the incubated adipocytes in a solution with osmolarity of 300 mOsm/L and of 150 mOsm/L in the presence of TDC, at a concentration of 500 μM versus the incubation time.

FIG. 4 is a graph illustrating the number of adipocytes versus time for adipocytes incubated in:
an aqueous saline solution with osmolarity of 300 mOsm/L,
an aqueous saline solution with osmolarity of 150 mOsm/L,
an aqueous saline solution with osmolarity of 150 mOsm/L, comprising TDC at a concentration of 500 μM.

FIG. 5 is a graph illustrating the number of adipocytes versus time for adipocytes incubated in:
an aqueous saline solution with osmolarity of 300 mOsm/L,
an aqueous saline solution with osmolarity of 90 mOsm/L,
an aqueous saline solution with osmolarity of 90 mOsm/L comprising TDC at a concentration of 500 μM.

FIG. 6 is a graph illustrating the number of adipocytes versus time for adipocytes incubated in:
an aqueous saline solution with osmolarity of 300 mOsm/L,
an aqueous saline solution with osmolarity of 300 mOsm/L, comprising TDC at a concentration of 500 μM,
an aqueous saline solution with osmolarity of 150 mOsm/L, comprising TDC at a concentration of 500 μM,
an aqueous saline solution with osmolarity of 90 mOsm/L, comprising TDC at a concentration of 500 μM.

FIG. 7 is a graph illustrating the activity of LDH versus time upon incubation of adipocytes in respectively:
a solution with osmolarity of 300 mOsm/L,
a solution with osmolarity of 150 mOsm/L,
a solution with osmolarity of 150 mOsm/L, comprising TDC at a concentration of 500 μM.

FIG. 8 is a graph representing the number of adipocytes present in the culture medium versus time for adipocytes incubated in:
an aqueous saline solution with osmolarity of 150 mOsm/L,
an aqueous saline solution with osmolarity of 150 mOsm/L comprising TDC at a concentration of 500 μM,
an aqueous saline solution with osmolarity of 150 mOsm/L comprising Pluronic® P85 at a concentration of 0.05%,
an aqueous saline solution with osmolarity of 150 mOsm/L comprising Pluronic® P85 at a concentration of 0.05% and TDC at a concentration of 500 μM.

FIG. 9 is a graph illustrating the number of adipocytes present in the culture medium versus time for adipocytes incubated in:
an aqueous saline solution with osmolarity of 300 mOsm/L,
an aqueous saline solution with osmolarity of 300 mOsm/L comprising TDC at a concentration of 500 μM,
an aqueous saline solution with osmolarity of 300 mOsm/L comprising Pluronic® P85 at a concentration of 0.05%,
an aqueous saline solution with osmolarity of 300 mOsm/L comprising Pluronic® P85 at a concentration of 0.05% and TDC at a concentration of 500 μM.

FIG. 10 is a graph representing the activity of LDH released during complete lysis of the adipocytes versus the concentration and nature of the poloxamer present in an aqueous saline solution with osmolarity of 300 mOsm/L, and this after one hour at 37° C.

FIG. 11 is a graph representing the activity of LDH released during complete lysis of the adipocytes versus time and this depending on the selection of the poloxamer present in an aqueous saline solution with osmolarity of 300 mOsm/L at a concentration of 0.1%.

FIG. 12 is a graph illustrating the activity of LDH released during complete lysis of the adipocytes after one hour versus the concentration of DC biliary acid salts present in an aqueous saline solution with osmolarity of 300 mOsm/L.

FIG. 13 is a graph illustrating the activity of LDH released during complete lysis of the adipocytes versus time of an aqueous saline solution with osmolarity of 300 mOsm/L which comprises DC as a biliary acid salt and at a concentration of 750 μM.

FIG. 14 is a graph illustrating the activity of LDH after one hour versus the osmolarity of an aqueous saline solution.

FIG. 15 illustrates a diagram of the activity of LDH after one hour, for:
A: an aqueous saline solution with osmolarity of 300 mOsm/L comprising 0.1% of P123,
B: an aqueous saline solution with osmolarity of 300 mOsm/L comprising 750 μM of DC,
C: an aqueous saline solution with osmolarity of 160 mOsm/L,
D: an aqueous saline solution with osmolarity of 160 mOsm/L comprising 750 μM of DC;
E: an aqueous saline solution with osmolarity of 160 mOsm/L comprising 0.1% of P123,
F: an aqueous saline solution with osmolarity of 160 mOsm/L comprising 0.1% of P123 and 750 μM of DC.

### EXAMPLES

### A—Experimental Results Obtained from Murine Adipous Tissues

It should be noted that the examples of this part A relate to in vitro tests using murine adipous tissues; this means that the cells are immersed in the culture medium representative of an aqueous saline solution according to the invention. With lower osmolarity, the lysis of the adipocytes during an in vitro test would have been too rapid and therefore would not have been able to be evaluated by for example counting the adipocytes over time.

This is why, the osmolarity of this culture medium described hereafter had to be limited to values greater than or equal to 90 mOsm/L and used as a representative model of the situation in situ which is obtained upon applying the invention on adipous tissues in situ.

### Preparation of the Adipocytes:

Adipocytes were prepared from murine adipous tissues in the following way. Adipous tissues were sampled under aseptic conditions on anesthetized animals, and then finally cut and subjected to a dissociating enzymatic treatment with collagenase type II at 37° C. The incubation mixture was then centrifuged in order to remove the tissue debris. The adipocytes were purified by filtration under sterile conditions on a 160 μm nylon filter. After mild centrifugation, the adipocytes were washed from a phosphate buffer solution with pH 7.4 and, after counting, taken up again into a cell culture medium adapted from the type of Dubelco Modified Eagle's Medium or DMEM, and finally placed in sterile culture flasks and incubator at 37° C. adapted to the experiments.

Treatment of the Prepared Adipocytes with a Solution According to the Invention:

The thereby prepared adipocytes were tested for one hour on different aqueous saline solutions comprising compounds customarily used for cell culture, such as amino acids, antibiotics and with respectively:
normal osmolarity, i.e. 300 mOsm/L,
hypotonic osmolarity, i.e. 150 mOsm/L,
highly hypertonic osmolarity of 90 mOsm/L,

and into which was added:
either a biliary acid salt selected from: sodium deoxycholate (DC), sodium lithocholate (LC), sodium tauro-deoxycholate (TDC), sodium tauro-lithocholate (TLC), at concentrations varying from 100 μM to 750 μM,
or a poloxamer, at concentrations comprised between 0.02% and 1.5%,
or a combination of biliary acids and poloxamers at concentrations within the aforementioned ranges.

The conditions for applying the aforementioned parameters were established from experimental schemes.

The significance of the results obtained was studied by means of statistical methods for comparing averages, and by means of variance analysis (ANOVA), followed by suitable tests (Dunett's and Bonferroni's test for multiple comparisons).

### Examination of the Treated Adipocytes:

Determination of the Number of Cells and of their Diameter:

The fat cells were then examined in an inverted microscope under suitable conditions (identical magnifications ×200, count over 50 cells) in order to measure their diameter versus the exposure time. Photographs were taken at specific time intervals (at t=0, 15, 30 and 45 min), in order to determine the number of cells and the diameter of the adipocytes by automatic measurements. The measurement of the change in diameter of the adipocytes allowed the swelling of the adipocytes to be appreciated, therefore the efficiency of the treatment according to the invention to be appreciated with regard to the hyperhydration phenomenon.

Cell lysis was appreciated by the change in the number of adipocytes on the one hand and by the dosage of an intracellular enzyme released into the culture medium, lactico-dehydrogenase (abbreviated hereafter as LDH) on the other hand, expressed as a percentage with respect to the conditions for which cell lysis is complete (obtained in the presence of distilled water) and by comparison with the conditions of cell cultures.

Some of the results obtained from different combinations of the aforementioned parameters are presented hereafter.

### Effect of Hypotonicity Alone:

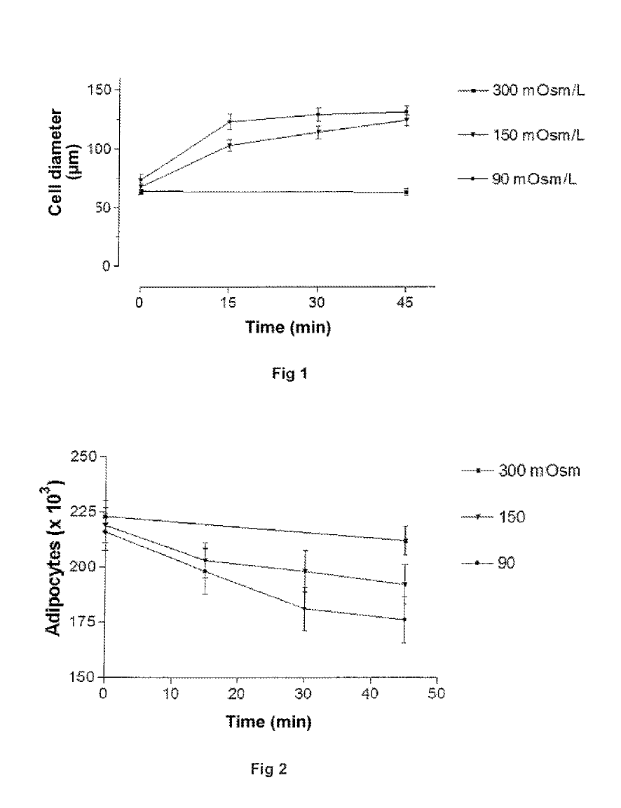

FIG. 1 is a graph illustrating the diameter (expressed in μm) of incubated adipocytes in the presence of aqueous saline solutions of the aforementioned osmolarities, i.e. isotonic osmolarity (300 mOsm/L) or hypotonic osmolarity (150 mOsm/L), and highly hypotonic osmolarity (90 mOsm/L), for different incubation times (expressed in minutes).

A gradual increase in the diameter of the adipocytes during their incubation in hypotonic solutions (150 and 90 mOsm/L) is noticed as compared with that during incubation in an isotonic solution. The increase in the diameter (comprised between 50 and 85%) is relatively rapid and reaches a plateau around 45 min. It is also found that these increases in diameter expressing cell swelling during incubation in aqueous saline solutions are faster for a highly hypotonic solution 90 mOsm/L than for a hypotonic solution of 150 mOsm/L).

FIG. 2 is a graph illustrating the time-dependent change in the number of adipocytes versus time for adipocytes incubated in the aforementioned aqueous saline solutions of the graph of FIG. 1, i.e. with osmolarities of 90, 150 and 300 mOsm/L.

It is seen from this graph of FIG. 2 that the smaller the osmolarity of the incubation solution of the adipocytes, the faster the number of adipocytes is decreased.

These first measurements of the hypotonicity effect on adipocytes and grouped in the graphs of FIGS. 1 and 2 will be used as a comparison with the measurements obtained from the association of an isotonic or hypotonic solution with a biliary acid salt.

### Cumulative Effect of the Salt of the Biliary Acid and of Hypotonicity on the Diameter of the Adipocytes:

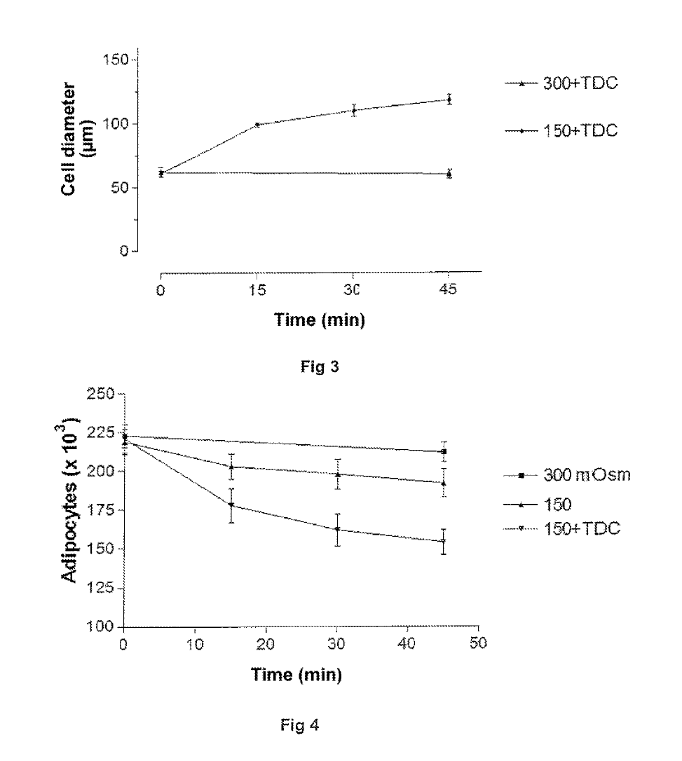

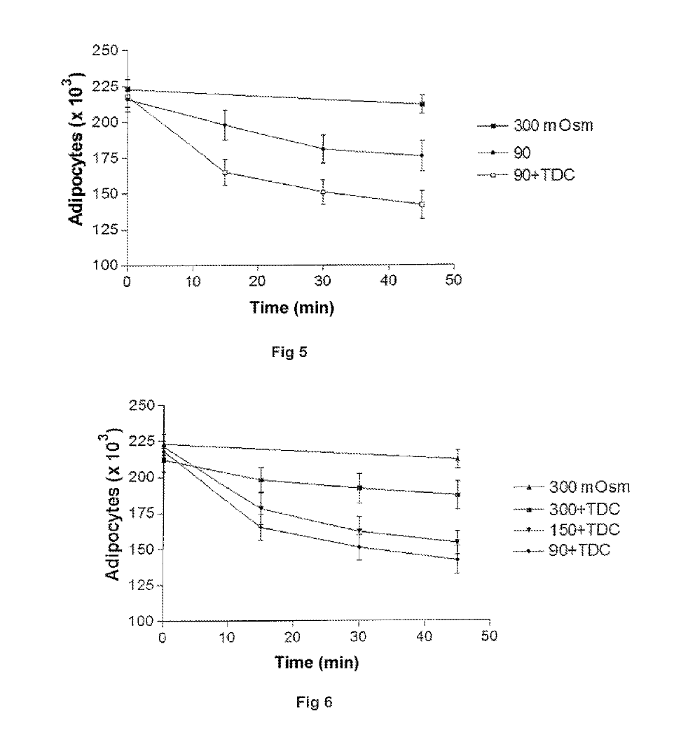

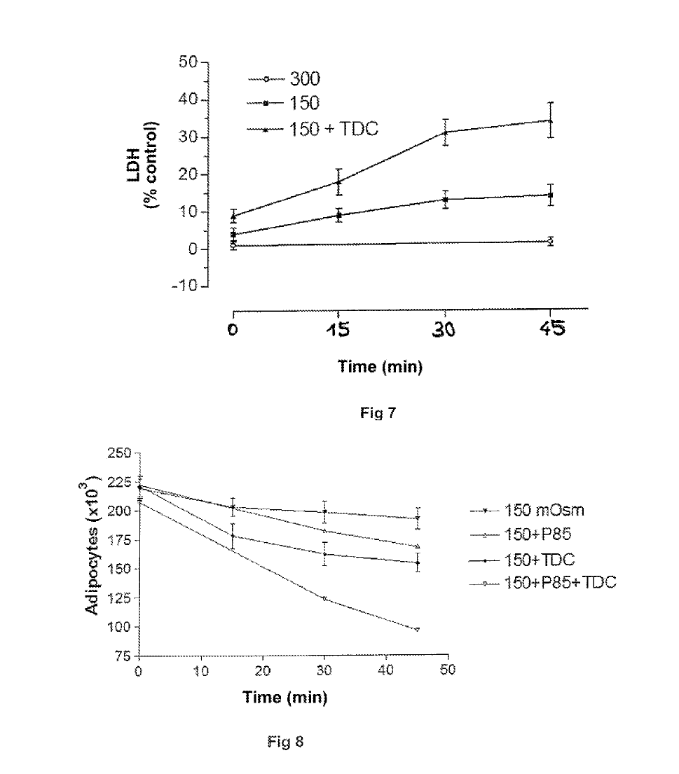

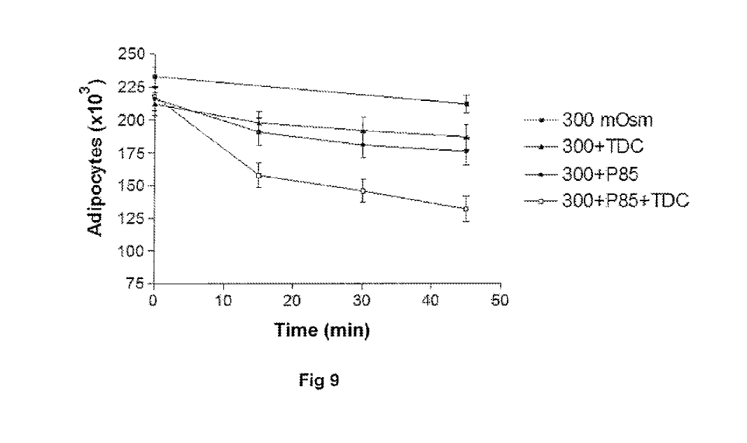

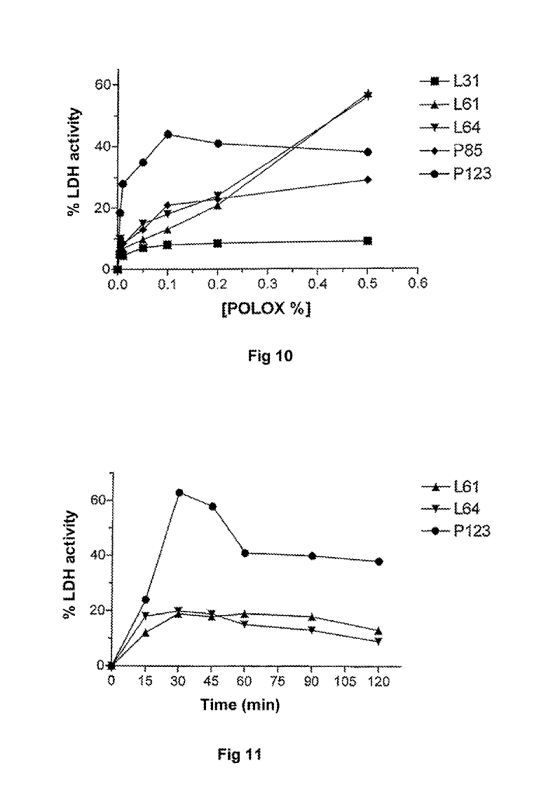

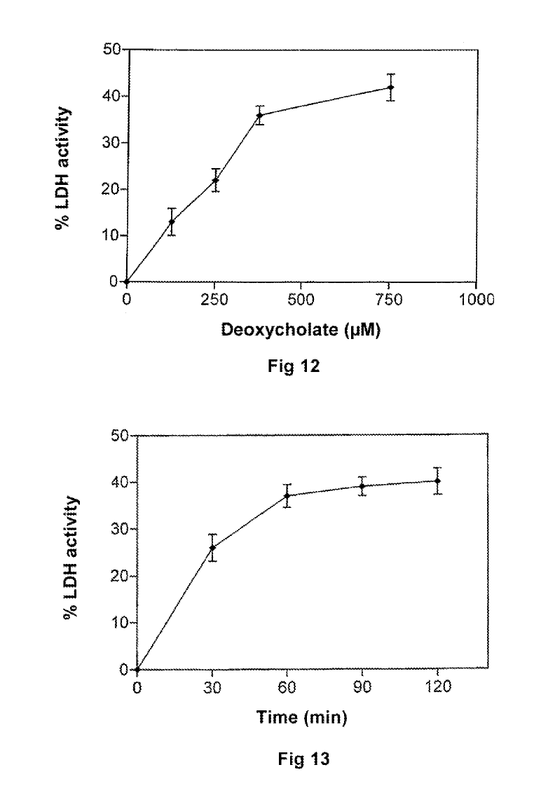

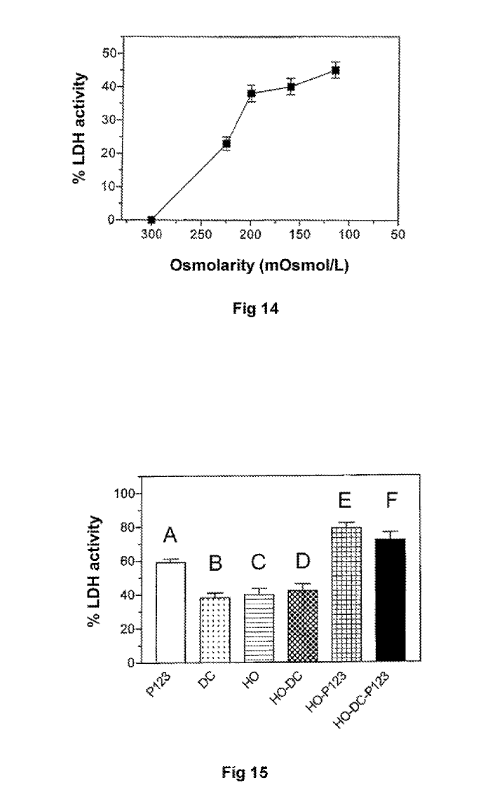

FIG. 3 is a graph illustrating the diameter of adipocytes incubated in an isotonic solution (300 mOsm/L) and in a hypotonic solution (150 mOsm/L) in the presence of TDC at a concentration of 500 μM, for different incubation times (expressed in minutes).

It is seen that the presence of biliary acid salts in an isotonic or hypotonic solution does not cause significant variation in the diameter of the adipocytes as compared with isotonic or hypotonic solutions alone.

### Cumulative Effect of a Biliary Acid Salt and of Hypotonicity on the Diameter of the Adipocytes:

FIG. 4 is a graph illustrating the number of adipocytes versus time for adipocytes incubated in:
an isotonic aqueous saline solution (300 mOsm/L),
a hypotonic aqueous saline solution (150 mOsm/L),
a hypotonic aqueous saline solution (150 mOsm/L), comprising TDC at a concentration of 500 μM.

It is seen that the number of adipocytes decreases very rapidly and in a more significant way when the hypotonic aqueous saline solution comprises a biliary acid salt as compared with a same aqueous saline solution but without this biliary acid salt. This shows the effect of the presence of a biliary acid salt in an aqueous saline solution according to the invention on the lysis of adipocytes.

FIG. 5 is a graph illustrating the number of adipocytes versus time for adipocytes incubated in:
an isotonic aqueous saline solution (300 mOsm/L),
a highly hypotonic aqueous saline solution (90 mOsm/L),
a highly hypotonic saline solution (90 mOsm/L) comprising DTC and at a concentration of 500 μM.

It is seen that the number or adipocytes decreases more rapidly and in a more substantial way when the highly hypotonic aqueous saline solution comprises a biliary acid salt as compared with a same aqueous saline solution without any biliary acid salt. This shows the effect of the presence of a biliary acid salt in an aqueous saline solution according to the invention on the lysis of adipocytes.

FIG. 6 is a graph illustrating the number of adipocytes versus time for adipocytes incubated in:
an isotonic aqueous solution (300 mOsm/L),
an isotonic aqueous saline solution (300 mOsm/L) comprising TDC at a concentration of 500 μM,
a hypotonic aqueous saline solution (150 mOsm/L) comprising TDC at a concentration of 500 μM,
a highly hypotonic aqueous saline solution (90 mOsm/L) comprising TDC at a concentration of 500 μM.

It is also seen that, when the isotonic aqueous saline solution (300 mOsm/L) contains a biliary acid salt, the number of adipocytes decreases unlike a same isotonic solution, but without this biliary acid salt, for which the number of adipocytes remains quasi constant over time, during the incubation.

It is also seen from this graph that more the aqueous saline solution (and comprising a biliary acid salt) is hypotonic, more the number of adipocytes decreases rapidly and in a more substantial way.

### Cumulative Effect of a Biliary Acid Salt and of Hypotonicity on the Activity of LDH:

FIG. 7 is a graph illustrating the activity of LDH versus time during the incubation of adipocytes in respectively:
an isotonic solution (300 mOsm/L),
a hypotonic solution (150 mOsm/L)n
a hypotonic solution (150 mOsm/L) comprising TDC at a concentration of 500 μM.

It is seen that the aqueous saline solution with osmolarity of 150 mOsm/L induces a moderate increase in the activity of LDH in the medium, the latter is much more increased upon adding TDC at a concentration of 500 μM to a same aqueous saline solution with osmolarity of 150 mOsm/L.

Cumulative Effect of Combination of Biliary Acid Salt with a Poloxamer TDC and of Hypotonicity on the Number of Adipocytes:

FIG. 8 is a graph illustrating the number of adipocytes present in the culture medium versus time for adipocytes incubated in:
a hypotonic aqueous saline solution (150 mOsm/L),
a hypotonic aqueous saline solution (150 mOsm/L) comprising TDC at a concentration of 500 μM,
a hypotonic aqueous saline solution (150 mOsm/L) comprising Pluronic® P85 at a concentration of 0.05%,
a hypotonic aqueous saline solution (150 mOsm/L) comprising Pluronic® P85 at a concentration of 0.05% and TDC at a concentration of 500 μM.

It is seen that the number of adipocytes decreases more and more rapidly when the hypotonic aqueous saline solution respectively contains:

a poloxamer such as Pluronic® P85,

a biliary acid salt such as TDC,

a mixture of Pluronic® P85 polaxomer and of a biliary acid salt TDC.

The graph of FIG. 8 particularly shows the synergistic effect of the combination of the biliary acid salt with a poloxamer as to the decrease in the number of adipocytes. Indeed, between 0 and 30 minutes, the number of adipocytes decreases very strongly, and this as compared with the other aforementioned tests and illustrated in this same graph.

Thus, this example shows the synergistic effect of the mixture of a biliary acid salt with a poloxamer in a hypotonic aqueous saline solution (150 mOsm/L) as regards the lytic activity of this solution.

FIG. 9 is a graph illustrating the number of adipocytes present in the culture medium versus time for adipocytes incubated in:
an isotonic aqueous saline solution (300 mOsm/L)
an isotonic aqueous saline solution (300 mOsm/L) comprising TDC at a concentration of 500 μM,
an isotonic aqueous saline solution (300 mOsm/L) comprising Pluronic® P85 at a concentration of 0.05%,
an isotonic aqueous saline solution (300 mOsm/L) comprising Pluronic® P85 and at a concentration of 0.05% and TDC at a concentration of 500 μM.

From this graph of FIG. 9, it is also seen that the number of adipocytes decreases more and more rapidly when the hypotonic aqueous saline solution respectively contains:
a biliary acid salt such as TDC,
a poloxamer such as Pluronic® P85,
a mixture of Pluronic® P85 poloxamer and of TDC biliary acid salt.

Thus, the graph of FIG. 9 also shows the synergistic effect of the combination of the biliary acid salt with a poloxamer as to the decrease in the number of adipocytes, and therefore the lytic effect of these aqueous solutions.

However, it is seen that the lytic effect is smaller for these isotonic aqueous saline solutions as compared with the one obtained from hypotonic solutions (and being the subject of graph 8).

For example, after 45 minutes, the number of adipocytes is of the order of 100×10³ for an aqueous saline solution with osmolarity of 150 mOsm/L comprising a mixture of TDC and Pluronic® P85 and of the order of 140×10³ for an aqueous saline solution with osmolarity of 300 mOsm/L comprising this same mixture.

Thus, by comparing the results illustrated on both graphs of FIGS. 8 and 9, the effect of osmolarity on the lytic activity of the aqueous saline solution according to the invention is seen. The smaller the osmolarity of the aqueous saline solution, the more significant is the lytic activity.

B—Experimental Results Obtained from Human Adipous Tissues:

II—Experimental Results Obtained from Human Adipous Tissues:

It should be noted that the examples of this 2^{nd} experimental part are relative to tests in vitro using human adipous tissues. Hypo-osmolarity is relatively delicate to test, since cells in culture have particular sensitivity because of their low density as compared with that of organized adipous tissues. Under the experimental condition described hereafter, it was not possible to test hypo-osmolarity below 115 mOsm/L because of the rapidity of action of this cell lysis mode (counting the adipocytes over time would not have been possible). Moreover, most experimental tests as to the selection of the membrane-weakening agent of the aqueous saline solution according to the invention were carried out with an osmolarity of 300 mOsm/L (isotonic solution).

Goals of this 2^{nd} Experimental Part:

The question was of determining the lytic effect of aqueous saline solutions according to the invention on human adipocytes in culture.

To do this, adipocytes were incubated for durations comprised between 0 and 2 hours, in aqueous saline solutions according to the invention, and for which the concentrations of poloxamers and of biliary acid salts were increasing and comprised between 0.005 and 0.5% and 125 and 750 nM respectively.

The efficiency of adipocyte lysis was appreciated by the measurement of the activity of a cytosolic enzyme lactico dehydrogenase (LDH), released in the culture medium and expressed as a percentage of the total lysis.

### Technical and Methodological Aspects:

The cells were derived from a line of human adipocytes (SW872) stemming from a liposarcoma deposited at the American Cell Bank of the National Institute of Health (NIH).

The adipocytes were cultivated in the following medium: DMEM (Dubelco's modified Eagle medium) with a high glucose content (4.5 g/L) which contained pyruvate, antibiotics (penicillin/streptomycin), a supplement of amino acids and 25% of Ham-F12 medium.

To this culture medium was added 10% of fetal calf serum for sustaining and multiplying the adipocytes.

It should be noted that the experiments were then conducted in the presence of 1% fetal calf serum in order to minimize basal LDH activity likely to be present in the serum.

The biliary acid salts were from SIGMA-ALDRICH.

The poloxamers were from BASF USA. Within the scope of this 2^{nd} experimental part, the following poloxamers were used: L31, L61, L64, P85, P123.

The salts of biliary acids and/or the poloxamers were put into solution in the aforementioned culture medium under sterile conditions. Suitable diluted aqueous saline solutions were made from parent solutions at 2% for poloxamers and 3 mM for the biliary acid salt.

### General Organization of the Procedure:

The adipocytes were cultivated under sterile conditions (plastic dishes, incubator with 5% CO₂, 37° C.) in suitable specific media and used for less than 10 passages oso as to conduct experiments on primary cells, i.e. close to the original adipous tissues. Cell counts were carried out in Mallassez cells and the viability was appreciated by exclusion of the blue dye Trypan.

After re-suspending them under the action of trypsin, the adipocytes were incubated in sterile 96-well plates. For experiments requiring significant amounts of cells, the preliminary incubations were conducted in sterile 24-well plates, and then transferred into 96-well plates (round bottom) for centrifugation, in order to efficiently separate entire cells from lyzed cells having released their enzymatic content then again found in the supernatant. An aliquot (100 μL) of the latter was then transferred into another 96-well plate adapted to dosages (flat bottom). After addition of the mixture of corresponding reagents (kit BioChain #K6330400), LDH activity was then measured (after incubation for 40 minutes with intermittent orbital stirring) in a computerized plate reader (MR5000/Biolinx, Dynatech) at the wavelength of 490 nm corrected from absorbance at 630 nm. The results are expressed as a percentage of the total lysis carried out in the presence of detergent (1% Triton X100).

It should be noted that no obvious contamination or cell viability problem was seen upon cultivating adipocytes; the latter remained constantly greater than 97%. There was no problem of solubilization of the compounds in the culture medium during the experiments and including during the storage at 4° C.

a) Activity of LDH According to the Poloxamer Present in the Aqueous Saline Solution as Well as to its Concentration:

FIG. 10 is a graph illustrating the activity of the LDH released during complete lysis of the adipocytes depending on the concentration (from 0 to 0.5%) and on the nature of the poloxamer present in an aqueous saline solution with osmolarity of 300 mOsm/, and this after one hour at 37° C. the tested poloxamers were L31, L61, L64, P85 and P123.

It is seen that L31 has little effect, the activity of LDH remaining always less than 10% and this, whatever the concentration of this poloxamer. It is found that the activity of LDH increases proportionally with the increase in the L61 and L64 concentration. At a concentration of 0.5% of L61 or L64, the activity of LDH rises to 56.5%. For the poloxamers P85 and P123, the lytic effects reach plateaus. It is however noted that P123 has a significant effect and this even at low concentrations, and this effect is maintained at the high concentrations with activity of LDH close to 40%.

These experimental results show the fact that the poloxamers because of their physico-chemical properties have the property of lyzing human adipocytes in culture. The poloxamers P85, L64, L61 and P123 are particularly efficient; the poloxamer L31 prove to be less efficient under these experimental conditions.

For a concentration of 0.1%, the order of efficiency is found to decrease according to the poloxamers: P123>L64 L61>P85>L31.

For a larger concentration of 0.5%, the order of decreasing efficiency of the poloxamers is substantially different: L64=L61>P123>P85>L31.

Further, the lytic effects of certain poloxamers such as L61 and L64 prove to be dependent on their concentration, while a plateau effect is observed for the poloxamers P85 and P123.

These action differences do not seem to be connected to their HLB since the latter are different and poloxamers with close HLBs have very different behaviors (for example L64 and P85).

b) Activity of LDH Depending on the Poloxamer Present in the Aqueous Saline Solution as Well as Versus Time:

FIG. 11 is a graph illustrating the activity of LDH released during complete lysis of the adipocytes versus time (0 to 120 minutes), and this depending on the selection of the poloxamer (L61, L64 or P123) present in an aqueous saline solution with osmolarity of 300 mOsm/L at a concentration of 0.1%. The temperature of the medium was maintained at 37° C.

It is seen that the effects of these three poloxamers are rapid since a significant activity of LDH is obtained after 15 minutes of exposure. The effects stagnate or decrease over time depending on these poloxamers. It is found that P123 is maintained at a lysis close to 40%.

c) Activity of LDH Depending on the Concentration of the Biliary Acid Salt DC Present in the Aqueous Saline Solution:

FIG. 12 is a graph illustrating the activity of LDH released during complete lysis of the adipocytes after one hour depending on the concentration of biliary acid salt DC (from 0 to 750 μM) present in an aqueous saline solution with osmolarity of 300 mOsm/L.

A maximum lysis of 42±7% is seen, obtained with an incubation of one hour in the presence of the adipocytes.

d) Activity of LDH Versus Time of an Aqueous Saline Solution Comprising Biliary Acid Salt Dc at a Concentration of 750 Um:

FIG. 13 is a graph illustrating the activity of LDH released during complete lysis of the adipocytes versus time (0 to 120 minutes) of an aqueous saline solution with osmolarity of 300 mOsm/L which comprises DC as a biliary acid salt and at a concentration of 750 μM.

It is seen that after 30 minutes of incubation, the LDH activity rises to 26% and attains a value of 37% after 2 hours of incubation.

e) Activity of LDH Versus the Osmolarity of an Aqueous Saline Solution:

FIG. 14 is a graph illustrating the activity of LDH after one hour versus the osmolarity of an aqueous saline solution (the osmolarity varying between 300 and 115 mOsm/L).

A rise in the LDH activity close to 25% is seen for hypo-osmolarity lowered to 225 mOsm/L and attaining 45±6% for the lowest tested osmolarity, i.e. 115 mOsm/L.

f) Activity of LDH Depending on the Combination of Different Parameters (Nature of the Membrane-Weakening Agent, Osmolarity of the Aqueous Saline Solution:

FIG. 15 illustrates a diagram of the activity of LDH after one hour, for:
A: an aqueous saline solution with osmolarity of 300 mOsm/L comprising 0.1% of P123,
B: an aqueous saline solution with osmolarity of 300 mOsm/L comprising 750 μM of DC,
C: an aqueous saline solution with osmolarity of 160 mOsm/L,
D: an aqueous saline solution with osmolarity of 160 mOsm/L comprising 750 μM of DC,
E: an aqueous saline solution with osmolarity of 160 mOsm/L comprising 0.1% of P123,
F: an aqueous saline solution with osmolarity of 160 mOsm/L comprising 0.1% of P123 and 750 μM of DC.

A significant lytic activity is thus seen, of the order of 59±5% of the poloxamer P123 as compared with the one obtained with a biliary acid salt DC at a concentration of 750 μM (38±5%). Hypo-osmolarity alone (160 mOsm/L) entails an LDH activity of the order of 40±8%. Further, the synergistic effect is found, obtained by combining hypo-osmolarity with the presence of poloxamer P123 with obtaining an LDH activity rising to 79±7%. It is moreover noted that the addition of DC to a hypo-osmolar aqueous saline solution does not induce additional lysis, since the activity of LDH is close to 72±8%.

The experimental results described above, certainly obtained under conditions (i.e. in vitro and therefore for doing this, a suitable osmolarity) different from those of the application of the aqueous saline solution according to the invention, tend to support the efficiency of this solution when it is applied in vivo.

1-14. (canceled)

15. An aqueous saline solution with osmolarity comprised between 50 mOsm/L and 170 mOsm/L, preferably between 50 and 100 mOsm/L, wherein it further comprises at least one membrane-weakening agent.

16. The aqueous saline solution according to claim 15, wherein said at least one weakening agent is selected from ionic detergents, non-ionic detergents, zwitterionic detergents and derivatives of glycerol.

17. The aqueous saline solution according to claim 16, wherein said at least one membrane-weakening agent is selected from alkyl glucosides, glucamides, polyoxiethylene, biliary acid salts.

18. An aqueous saline solution with osmolarity comprised between 50 mOsm/L and 170 mOsm/L, preferably between 50 and 100 mOsm/L according to claim 15, wherein it further comprises as a membrane-weakening agent at least one biliary acid salt and/or at least poloxamer of structural formula (I): 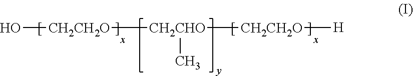
which comprises a hydrophilic portion and a hydrophobic portion, with x and y being integers, and selected so that the hydrophilic portion represents between 10 and 80% by weight of the poloxamer, preferably between 30 and 50% and the HLB (hydrophilic/lipophilic balance) is less than 35, preferably less than 20.

19. The aqueous saline solution according to claim 17, wherein the concentration of biliary acid salt is comprised between 50 μm and 3,000 μM, preferably between 50 and 1,000 μM, still more preferentially between 150 and 750 μM and/or the concentration of poloxamer is comprised between 0.001% and 5% by weight/volume, preferably between 0.1% and 1% by weight/volume.

20. The aqueous saline solution according to claim 17, wherein said at least biliary acid salt is selected from sodium deoxycholate (DC), sodium deoxycholate (TDC), sodium lithocholate (LC) and sodium tauro-lithocholate (TLC).

21. The aqueous saline solution according to claim 18, wherein said at least one poloxamer is selected from poloxamers of formula (I) which have:
a hydrophilic portion representing 40% of poloxamer and the HLB is 16,
a hydrophilic portion representing 10% of the poloxamer and the HLB is 3,
a hydrophilic portion representing 20% of the poloxamer and the HLB is 7,
a hydrophilic portion representing 30% of the poloxamer and the HLB is 11,
a hydrophilic portion representing 40% of the poloxamer, and the HLB is 15,
a hydrophilic portion representing 50% of the poloxamer and the HLB is 14,
a hydrophilic portion representing 40% of the poloxamer and the HLB is 14,
a hydrophilic portion representing 50% of the poloxamer and the HLB is 16,
a hydrophilic portion representing 30% by weight of the poloxamer, and the HLB is 8.

22. The aqueous saline solution according to claim 21, wherein the poloxamer is a poloxamer of formula (I) which has a hydrophilic portion representing 30% by weight of the poloxamer, and the HLB is 8, and preferably Pluronic® P 123.

23. The aqueous saline solution according to claim 15, wherein it comprises at least one salt selected from NaCl at a concentration comprised between 10 and 25 mOsm/L, NaHCO₃ at a concentration comprised between 5 and 15 mOsm/L, MgCl₂ at a concentration comprised between 0.5 and 5 mOsm/L.

24. The aqueous saline solution according to claim 15, wherein it further comprises at least one substance selected from adrenalin, an anaesthetic such as lidocaine.

25. The aqueous saline solution according to claim 24, wherein the adrenalin has a concentration comprised between 0.5 and 2 mg/l and the lidocaine a concentration comprised between 1 and 5% by weight/volume.

26. The aqueous saline solution according to claim 15, wherein it is without any potassium.

27. The aqueous saline solution according to claim 15 configured for obtaining a drug intended for treating cellulitis, steatomery as well as lipoma.

28. A cosmetic composition wherein it comprises an aqueous saline solution according to claim 15.

29. A cosmetic composition according to claim 28 for non-therapeutic treatment of cellulitis, steatomery, or lipoma.

30. A method for aesthetical treatment consisting in the administration of an aqueous saline solution according to claim 15, wherein said administration is performed by an intra-fat injection method.

31. A method for aesthetical treatment according to claim 30, wherein the administration is performed as follows:
after local anaesthesia by small epidermal injections of the aqueous saline solution according to claim 10 which contains lidocaine,
the aqueous saline solution according to claim 10 which contains lidocaine is injected subcutaneously into the targeted adipous tissue at different injection depths and always through the same injection point.

32. A method for aesthetical treatment according to claim 31, wherein said method is completed by an ultrasonic treatment.
